(19) United States
(12) Patent Application Publication
(10) Pub. No.: US 2026/0237522 A1
(43) Pub. Date: Aug. 13, 2026

(54) BATCH PROCESSING OF MEDICAL STUDIES

(71) Applicant: Atropos Health, Inc., New York, NY (US)

(72) Inventors: Rehan N. Chawdry, Dublin, OH (US); Uday Reddy, San Mateo, CA (US); Vladimir Polony, Novato, CA (US); Brigham Hyde, Purchase, NY (US); Saurabh Gombar, San Diego, CA (US); Neil M. Sanghavi, Dublin, OH (US)

(21) Appl. No.: 19/538,448

(22) Filed: Feb. 12, 2026

Related U.S. Application Data
(60) Provisional application No. 63/757,646, filed on Feb. 12, 2025.

(51) Int. Cl. G16H 50/70; G16H 50/20

(52) U.S. Cl. CPC G16H 50/70; G16H 50/20

(57) ABSTRACT

A method for batch processing of medical studies can include receiving, by a computing system, input describing a plurality of medical studies, wherein the input includes a query template and variable parameters with corresponding values. The method can include generating a plurality of jobs based on the query template and combinations of variable parameters, wherein each job corresponds to a distinct combination of parameter values. The method can include executing a subset of jobs to produce results. The method can include performing statistical analysis on results to identify jobs that produced statistically significant results. Based on the statistical analysis, the method can include determining parameters for subsequent jobs, wherein the determining comprises modifying subsequent jobs or generating additional jobs to further explore parameters associated with statistically significant results.

### CROSS-REFERENCE TO RELATED APPLICATIONS

This application claims priority to U.S. Provisional Application No. 63/757,646, titled “BATCH PROCESSING OF MEDICAL STUDIES,” filed February 12, 2025, which is hereby incorporated by reference in its entirety.

### FIELD OF INVENTION

The present disclosure relates to medical data analysis and retrospective study systems, and more particularly to batch processing systems and methods for automating the creation, execution, and optimization of multiple medical studies using parameterized queries and statistical analysis to identify significant results.

### BACKGROUND

Retrospective medical studies utilizing real-world evidence from electronic health records, claims data, and other sources can provide valuable insights for improving patient care. However, conducting such studies presents numerous challenges, including the complexity of working with heterogeneous data sources, the difficulty of accounting for confounding variables, and the substantial effort involved in designing and executing multiple study variants to explore different patient populations, treatments, and outcomes. When researchers wish to investigate how various factors influence health outcomes, the number of potential study configurations can grow rapidly, making manual definition and execution of individual studies impractical. Accordingly, improvements in systems and methods for conducting medical studies are desired.

### BRIEF DESCRIPTION OF FIGURES

The technologies described herein will become more apparent to those skilled in the art from studying the Detailed Description in conjunction with the drawings. Embodiments or implementations describing aspects of the invention are illustrated by way of example, and the same references can indicate similar elements. While the drawings depict various implementations for illustration, those skilled in the art will recognize that alternative implementations can be employed without departing from the principles of the present technologies. Accordingly, while specific implementations are shown in the drawings, the technology is amenable to various modifications.

FIG. 1 is a drawing that illustrates an example user interface 100 for batch management according to some implementations.

FIG. 2 is a drawing that illustrates an example batch configuration user interface 200 according to some implementations.

FIG. 3 is a drawing that illustrates an example user interface for managing jobs in a batch according to some implementations.

FIG. 4 is a drawing that schematically illustrates multivariable jobs.

FIG. 5 is a flowchart that illustrates an example batch processing process according to some implementations.

FIG. 6 is a flowchart that illustrates an example process for optimizing jobs in a batch according to some implementations.

FIG. 7 is a flow chart that illustrates another example process for optimizing jobs in a batch according to some implementations.

FIG. 8 is a flowchart that illustrates an example batch processing implementation.

FIG. 9 is a block diagram depicting an embodiment of a computer hardware system configured to run software for implementing one or more of the systems and methods described herein.

### DETAILED DESCRIPTION

Although several implementations, embodiments, examples, and illustrations are disclosed below, it will be understood by those of ordinary skill in the art that the scope of the present disclosure extends beyond the specifically disclosed implementations, embodiments, examples, and illustrations and includes other uses of the inventions and obvious modifications and equivalents thereof. Implementations are described with reference to the accompanying figures, wherein like numerals refer to like elements throughout. The terminology used in the description presented herein is not intended to be interpreted in any limited or restrictive manner simply because it is being used in conjunction with a detailed description of certain specific implementations. In addition, implementations can comprise several novel features, and no single feature is necessarily essential or solely responsible for its desirable attributes.

### Overview

Retrospective studies are a powerful tool for medicine and can provide insights that can be used to improve care for groups ranging from entire populations to individual patients. However, there are many difficulties associated with retrospective studies, which can limit their use. For example, obtaining and analyzing data can be challenging, data may be incomplete, incorrect, inconsistent, and/or of poor quality, confounding variables can make it difficult to draw meaningful inferences, and so forth. The approaches described herein can enable physicians and other medical professionals to more easily make use of retrospective studies. Retrospective studies can utilize real-world evidence (RWE), such as data from published peer-reviewed studies, electronic health records, claims data, pharmacy data, public health tracking databases, and so forth. Real-world evidence can, in some cases, provide a richer set of data than clinical trials. For example, real-world evidence can be used to trace health outcomes, interventions, treatments, and so forth across hundreds, thousands, or even millions of individuals over time periods ranging from days to decades, while clinical trials are typically limited in time and include a much smaller population. Real-world evidence can also avoid or reduce ethical issues commonly associated with clinical trials, such as limiting one group’s access to a potentially life-saving treatment.

The approaches described herein can utilize health record data such as electronic medical records, pharmacy data, and insurance data to conduct studies. In some implementations, the platform incorporates data from alternative sources beyond traditional electronic health records. For example, the platform may integrate data from wearable devices and fitness trackers, which can provide measurements of heart rate, activity levels, sleep patterns, and other health indicators. The platform may incorporate patient-reported outcomes collected through surveys or mobile applications, capturing subjective measures of health status, quality of life, and treatment satisfaction. The platform may integrate social determinants of health data, such as information about housing stability, food security, transportation access, and neighborhood characteristics, which can significantly influence health outcomes. In some implementations, the platform may incorporate environmental data, such as air quality indices, pollen counts, or weather patterns, which may be relevant for studies of respiratory conditions, allergies, or other environmentally-influenced health outcomes.

In some implementations, the platform may address data quality issues through various automated and semi-automated techniques. For example, the platform may identify missing data and apply appropriate imputation methods, such as mean imputation, multiple imputation, or machine learning-based imputation, depending on the nature of the missing data and the study requirements. The platform may detect and flag potential outliers or erroneous values, enabling researchers to review and address data quality issues before analysis. The platform may identify inconsistencies across data sources, such as conflicting diagnoses or implausible sequences of events, and provide tools for reconciling these inconsistencies. In some implementations, the platform may assign data quality scores to records or data elements, enabling researchers to filter or weight data based on quality considerations.

In some implementations, the platform may provide specialized support for analyzing longitudinal data and temporal relationships. For example, the platform may enable researchers to define time windows relative to index events, such as analyzing outcomes within 30 days, 90 days, or one year of a treatment initiation. The platform may support the analysis of treatment sequences, enabling researchers to study how patients transition between different treatments over time and how these transitions affect outcomes. The platform may provide tools for analyzing time-varying covariates, accounting for the fact that patient characteristics such as weight, blood pressure, or medication use may change over the course of a study period. In some implementations, the platform supports the analysis of competing risks, accounting for the possibility that patients may experience multiple types of events that affect the interpretation of study results.

Healthcare data is frequently stored in disparate data silos across various locations and systems. Patient information, test results, imaging data, diagnostic data, pharmacological information, electronic health records, and the like may be produced and stored in one or more proprietary formats as text, images, video, multimedia, and the like. Records may be electronically stored in disparate locations in various hospital departments, doctors' offices, and with outside providers in a variety of structured, semi-structured, and unstructured formats, making the collection and analysis of an individual’s entire record, let alone collections of records from multiple individuals, difficult. These disparate storage systems can make it challenging to deduce cross-correlations and can prevent generalized applications of machine learning to the collective data. Further, each silo may have different security and access requirements, increasing the level of complexity and difficulty in accessing even individual records.

The complexity of medical coding systems presents additional challenges for retrospective studies. The healthcare landscape has grown increasingly complex, with the number of disease conditions known to clinicians ever increasing as scientific discovery details a more granular understanding of pathogenesis, genetics, and sub-segmentation of previously less-understood conditions. The coding granularity by which such conditions are reflected within medical record documentation is similarly rising, as exemplified by the transition from ICD-9 (containing approximately 14,000 diagnosis codes) to ICD-10 (containing approximately 68,000 diagnosis codes) standards. Different healthcare systems may use different coding schemes, such as ICD-9, ICD-10, SNOMED CT, RxNorm, LOINC, or proprietary coding systems. A researcher attempting to identify patients with a particular condition may need to account for multiple coding schemes, historical changes in coding practices, and variations in how different institutions or even individual providers apply codes. For example, a study of diabetes patients may need to consider numerous ICD-10 codes (E08-E13 and their subcodes), legacy ICD-9 codes (250.xx), SNOMED CT concepts, and institution-specific or provider-specific coding practices. The batch processing approaches described herein can help address these challenges by enabling researchers to define studies that account for multiple coding schemes and by automatically mapping between different terminologies using phenotype libraries or other reference data.

Confounding variables present significant challenges in retrospective studies. For example, socioeconomic factors can influence both health behaviors and access to care, making it difficult to isolate the effects of specific treatments. Geographic factors can affect exposure to environmental hazards, access to healthcare facilities, and the prevalence of certain conditions. Seasonal variations can impact the incidence and severity of certain diseases. By running multiple study variants across different populations, time periods, and geographic regions, the batch processing approaches described herein can help researchers identify and account for confounding variables. In some implementations, the platform may automatically suggest potential confounding variables based on the study design and available data. In some implementations, the platform may perform sensitivity analyses to assess the robustness of findings across different assumptions about confounding variables.

Health outcomes are often influenced by a multitude of factors, such as underlying health conditions that can exacerbate the effects of an illness or treatment, genetic predispositions that can impact how people respond to diseases and treatments, environmental conditions such as exposure to hazardous materials or polluted air, lifestyle factors such as diet and exercise, and so forth. There are often confounding variables that can obscure the true relationships between conditions, treatments, and outcomes. For example, older individuals may tend to have worse outcomes due to age-related vulnerabilities as opposed to a condition itself. The presence of comorbidities can complicate analysis and make it difficult to determine causality or if there even is a true relationship between, for example, a treatment and an outcome, as it may be unclear whether observed effects or outcomes are due to a condition under investigation or other diseases or conditions.

Timing can also play an important role. For example, some health effects of a disease or treatment may not appear immediately, instead developing over weeks, months, years, or even decades. In the real world, as opposed to highly controlled studies, individuals often receive multiple treatments or interventions over relevant time periods, making it difficult to isolate the effects of each treatment or intervention. These treatments or interventions may or may not be directly related to a condition under investigation. For example, an individual may take one medication for high blood pressure during a first period of time and switch to another medication for high blood pressure at a later time. As an example of an indirect relationship, a patient may take a corticosteroid for arthritis. In a study about diabetes treatments, it can be important to know which patients in the study are also taking corticosteroids, which can cause increases in blood glucose levels. In some cases, the impacts of other treatments, interventions, combinations of treatments, etc., may be known. However, in some cases, such impacts may not be known. Advantageously, by using large volumes of data covering many patients and by running multiple study variants, the approaches described herein can provide robust results despite these impacts and, in some cases, may even lead to the discovery of previously-unknown impacts.

Data quality and completeness can also make it difficult to identify relationships in medical data. For example, there may be missing or incomplete data, which can lead to biased or inaccurate results. Different physicians, medical facilities, countries, and so forth may record information differently. Some physicians may order testing (e.g., blood tests) more frequently or order more complete panels than other physicians. In some cases, certain demographics may undergo less testing, less frequent follow-up visits, etc., than other demographics. For example, in countries where medical care is largely privatized, lower-income individuals may be less likely to seek out treatment or may seek to limit their out-of-pocket costs by doing fewer follow-up visits or opting out of tests that are not strictly necessary. Patients with better insurance may undergo more comprehensive or frequent medical care than those with more limited coverage or higher out-of-pocket costs. This can lead to biases and skews in medical information, as the information that is available may not accurately reflect the population as a whole.

Real world patient data is often at least in part self-reported. Patients may offer inconsistent or inaccurate accounts. For example, a diabetes patient may tell their physician that they are following a low sugar diet while actually drinking sugary sodas, or a patient with high blood pressure may report that they take their medication every day while actually routinely missing doses. Patients may conceal facts that they find embarrassing or that they feel like they would be judged for, such as their smoking or drinking habits. Even when patients try to provide honest, accurate accounts, they may not remember past events accurately or may not realize that an event is relevant and thus may not tell their physician about it. Some physicians may conduct a more in-depth discussion with their patients than others. In some cases, the same provider may provide different levels of background discussion with different patients. Differences in how data is collected can also affect quality, usability of data, and so forth.

In some cases, study designs may be biased. For example, there can be selection bias, recall bias, or observer bias. That is, a population for a study may not be representative of the general population, study participants may not accurately recall events, or observers (e.g., researchers) may be influenced by their own expectations. The approaches herein can alleviate some of these problems by performing multiple analyses on a large data set, without reliance on a relatively small study population but instead utilizing large-scale real-world evidence.

In some implementations, the platform utilizes automated cohort generation to help mitigate study design bias. For example, the platform can automatically identify appropriate control groups based on the characteristics of the treatment group, using techniques such as propensity score matching to create balanced comparison groups. In some implementations, the platform suggests inclusion and/or exclusion criteria based on the study objectives and available data. In some implementations, the platform automatically identifies potential sources of selection bias and suggests modifications to the study design to address them.

In some implementations, the platform utilizes machine learning or artificial intelligence to identify patterns across multiple study results. For example, the platform can employ a clustering algorithm to group studies with similar outcomes, enabling researchers to identify common factors that contribute to particular results. In some implementations, the platform uses natural language processing to analyze study descriptions and results, identifying relationships that may not be apparent from numerical data alone. In some implementations, the platform utilizes a deep learning model trained on historical study data to predict which study configurations are most likely to yield statistically significant results. These machine learning approaches can help researchers navigate the vast space of possible study configurations and focus their efforts on the most promising avenues of investigation.

As described herein, it can be difficult to attribute specific health effects to a specific cause, such as a particular treatment or disease. For example, effects can be directly caused by a treatment or disease, indirectly caused by the treatment or disease, or may be unrelated to the treatment or disease. By performing a larger number of studies that test more variables and/or combinations of variables, medical professionals can gain a better understanding of true causes, correlations, and so forth. Advantageously, the platform as described herein can, in some implementations, provide analytical functionality that can be used to identify true health drivers. In some implementations, the platform automatically analyzes the results of multiple studies to determine factors, treatments, etc., that have a significant impact on health outcomes.

As an example, consider COVID-19. COVID-19 can cause a wide variety of symptoms ranging from mild (like a cold) to severe and potentially lethal. Many individuals recover from COVID-19, while others experience prolonged effects. Different individuals receive different treatments, such as antivirals, steroids, oxygen therapy, etc., which can have different effects and different interactions. In the context of COVID-19, many took medications or used folk treatments that, while not effective for treating COVID-19, may nonetheless have impacts that can complicate analysis and make it difficult to draw sound conclusions. Many individuals received multiple different treatments, making it difficult to identify which treatments were effective, which were not, what side effects are associated with specific treatments, and so forth.

Continuing with the COVID-19 example, suppose a researcher wanted to evaluate the efficacy of various vaccines in preventing contraction of COVID-19 or preventing severe COVID-19. There are many different vaccines, and different vaccines were more or less available in different regions at different times. In the case of multi-dose vaccines, different people waited different amounts of time before receiving an additional dose. Different variants of the COVID-19 virus were prevalent in different regions at different times. Different groups of people took more or fewer precautions. These and other factors make it very difficult to determine vaccine efficacy. For example, a poorer, denser population with limited access to healthy food and sanitation but which is highly vaccinated may nonetheless have worse outcomes than another group of people who refused to be vaccinated but who live in rural areas with ready access to quality food, water, and shelter. Someone living in a dense urban environment may be more likely to contract COVID-19 despite being vaccinated and taking precautions than another individual who is not vaccinated and takes few or no precautions but whose lifestyle naturally limits their interactions with other people and potential exposure.

As another example, consider diabetes treatment efficacy studies. Diabetes is a complex condition with multiple subtypes (Type 1, Type 2, gestational, etc.), and treatment approaches vary widely based on disease progression, patient characteristics, and comorbidities. A researcher investigating the comparative effectiveness of different diabetes medications may need to account for factors such as baseline HbA1c levels, duration of disease, presence of complications (e.g., nephropathy, retinopathy, neuropathy), concurrent medications, dietary habits, exercise patterns, and socioeconomic factors that affect adherence. Using the batch processing approaches described herein, a researcher can define a base study and systematically vary parameters such as patient age groups, disease duration, baseline severity, and concurrent conditions to generate a comprehensive set of studies that explore the treatment landscape.

Cancer treatment outcome studies present similar challenges. Cancer encompasses hundreds of distinct diseases, each with its own molecular characteristics, staging systems, and treatment protocols. A researcher studying the effectiveness of immunotherapy for lung cancer may need to consider tumor histology, molecular markers (e.g., PD-L1 expression, EGFR mutations, ALK rearrangements), disease stage, prior treatments, patient performance status, and numerous other factors. The batch processing approaches described herein can enable researchers to systematically explore these variables, running studies across different patient subgroups and treatment combinations to identify factors that predict treatment response.

Cardiovascular disease risk factor studies illustrate the value of batch processing for exploring complex, multi-factorial conditions. Cardiovascular disease risk is influenced by numerous factors such as blood pressure, cholesterol levels, smoking status, diabetes, family history, diet, exercise, stress, and genetic predispositions. These factors interact in complex ways, and their relative importance may vary across different populations. By running batches of studies that systematically vary the factors under investigation, researchers can develop a more nuanced understanding of cardiovascular risk and identify subpopulations that may benefit from targeted interventions.

The batch processing approaches described herein may be especially valuable for studying rare diseases. Rare diseases, by definition, affect small numbers of patients, making it difficult to accumulate sufficient data for statistically meaningful studies. However, by aggregating data across multiple institutions and running studies that explore different definitions of the patient population (e.g., varying diagnostic criteria, including related conditions), researchers may be able to identify larger cohorts suitable for analysis. In some implementations, the platform may automatically identify related conditions or phenotypes that could be included in a study to increase sample size while maintaining clinical relevance.

Understanding complex, multi-variable problems and gaining insights from available data is a daunting task. Health professionals such as physicians, clinical researchers, pharmaceutical researchers, and so forth often struggle to make sense of large volumes of clinical data, claims data, published research papers, and so forth. Practicing physicians may find themselves especially constrained as they may be busy seeing patients and have little time to devote to researching questions. Even researchers who are focused on conducting research may nonetheless find it tedious and time-consuming to analyze health data, and the significant effort that may be put in can reduce the amount of research a health professional does, the depth or thoroughness of the research, and so forth. In some cases, a researcher may be unaware of certain features in available data, such as correlations or causal relationships, and may have no practical way of uncovering such features when there are complex relationships between different variables included in the data or confounding variables, conditions, etc., making analysis noisy and difficult to obtain reliable results.

When a health professional wishes to investigate a question, doing so can be a long and difficult process. For example, as described herein, finding data (e.g., electronic health record (EHR) data, claims data), finding published papers, analyzing data, and so forth can involve a considerable time commitment and may be outside the skillset of many medical professionals. As an example, a health professional who wishes to conduct a retrospective observational study (e.g., a study of past patients) can formulate a question, identify relevant patient populations, retrieve relevant data, perform statistical analysis on the data, and interpret the results. Each of these steps can present significant challenges.

Formulating a question can be difficult for a variety of reasons. For example, it can be important to determine an appropriate age group, control conditions (e.g., no treatment, a standard treatment, etc.), timeframes, and so forth. Identifying the patient population can be a significant undertaking. For example, a physician who wishes to perform analysis of patients with diabetes may search for patients whose health records include particular codes (e.g., ICD-10 codes) that correspond to diabetes. Data may come from a variety of sources, different coding schemes may be used, there may be many different codes that relate to diabetes, and so forth, which can make it difficult to identify the relevant patient population. A researcher may not be aware of all of the relevant diagnostic codes, procedure codes, and so forth, and thus may miss certain patients who should be included in the study. After determining relevant patients, additional challenges remain. Retrieving data can be difficult, as the data may be stored in different databases, have different formats and/or field names, and so forth.

Statistical data analysis can involve significant effort and is prone to user error. After conducting statistical analysis, a health professional can review the results and determine insights provided by the results (e.g., observing that a first intervention performs better than a second intervention in a particular population). Each of these steps can involve significant effort and require certain expertise. For example, a healthcare professional may be unfamiliar with how to transform data or write queries. Healthcare professionals may have limited knowledge of statistics, leading to poor analysis and poor or inaccurate interpretation of results. In some cases, performing such work may only be feasible for a limited set of health professionals, such as those affiliated with major pharmaceutical companies, medical schools, research institutions, and so forth, where they have access to individuals with expertise in data science, statistics, and so forth, or where their job roles allow them to develop such expertise. Yet, other healthcare professionals and their patients could obtain substantial benefits from being able to conduct data-driven investigations into prognoses, disease treatment, and so forth. Moreover, even dedicated researchers can benefit from thorough and accurate population definition, data analysis, and so forth. As described herein, there are many benefits to an analytical platform beyond retrieving and analyzing data (which are substantial benefits in their own right).

Querying heterogeneous data sources presents significant technical challenges. Different data sources may use different database technologies (e.g., relational databases, NoSQL databases, data lakes, etc.), different query languages (e.g., SQL, GraphQL, proprietary APIs), and different data models. Even when data sources use similar technologies, field names, data types, and coding conventions may differ. For example, one data source may store patient age as a date of birth, while another stores it as an integer representing years. One data source may use string codes for diagnoses, while another uses integer identifiers. The platform described herein may address these challenges by providing a unified query interface that abstracts away the underlying complexity of heterogeneous data sources. In some implementations, the platform may utilize a data integration schema that defines how data from different sources is mapped to a common representation. In some implementations, the platform may automatically transform data from different sources into a consistent format suitable for analysis.

In some implementations, the platform may provide natural language interfaces that enable users to define studies without needing to write queries in a formal query language. For example, a user may describe a study in plain English, such as "Compare outcomes for patients over 65 with Type 2 diabetes who received metformin versus those who received sulfonylureas." The platform may utilize natural language processing and large language models to interpret this description and generate the appropriate queries. In some implementations, the platform may engage in a dialogue with the user to clarify ambiguities and refine the study definition. In some implementations, the platform may suggest refinements or alternatives based on the available data and the user's apparent objectives.

In some implementations, the platform may provide simplified query builders that enable users to define studies through graphical user interfaces rather than text-based queries. For example, the platform may provide drag-and-drop interfaces that allow users to select patient populations, treatments, outcomes, and covariates from menus or palettes. In some implementations, the platform may provide templates for common study types (e.g., comparative effectiveness studies, safety studies, epidemiological studies) that users can customize for their specific needs. In some implementations, the platform may provide guided wizards that walk users through the process of defining a study, prompting them for necessary information and validating their inputs at each step.

In some implementations, users may specify study parameters through various alternative mechanisms. For example, users may upload spreadsheets or other structured files that define the parameters for multiple studies. Users may define parameters programmatically through APIs, enabling integration with other research tools and workflows. Users may define parameters through voice interfaces, enabling hands-free operation in clinical settings. Users may collaborate with other researchers to define parameters, with the platform providing tools for version control, commenting, and conflict resolution. These alternative mechanisms can make the platform accessible to a wider range of users and enable integration with diverse research workflows.

As described above, there are many considerations when designing a retrospective study. Often, a great deal of time and effort goes into designing a study. For example, different age groups, ethnicities, genders, combinations of treatments, combinations of diseases, socioeconomic factors, and so forth can be considered. A highly targeted study may produce more accurate or useful results for a particular group, but there may not be enough data available for a robust study, or there may not be sufficient resources available to justify conducting such a study. The difficulty of designing and running case studies can mean that only a limited number of studies are carried out.

As discussed herein, designing and carrying out case studies can be a highly complex task that requires significant expertise, but even if such expertise is present, researchers may still struggle to obtain meaningful or comprehensive results, as there can be many factors involved in a study, limitations to the available data, and so forth. Significant advancements have been made to enable a wider range of individuals to conduct meaningful studies without needing to have a great deal of expertise in coding or statistics. For example, techniques and improvements described in U.S. App. No. 61/612,925, filed 03/19/2012, U.S. App. No. 61/656,930, filed 06/07/2012, U.S. App. No. PCT/US13/32389, filed 03/15/2013, U.S. App. No. 13/943,588, filed 03/15/2013, U.S. App. No. 14/224,903, filed 03/25/2014, U.S. App. No. 16/911,098, filed 06/24/2020, U.S. App. No. 63/310,014, filed 02/14/2022, U.S. App. No. 63/315,989, filed 03/02/2022, U.S. App. No. 63/269,525, filed 03/17/2022, U.S. App. No. 63/269,526, filed 03/17/2022, PCT App. No. PCT/US23/62492, filed 02/13/2023, U.S. App. No. 18/168,263, filed 02/13/2023, U.S. App. No. 18/331,108, filed 06/07/2023, U.S. App. No. 63/596,594, filed 11/06/2023, and PCT App. No. PCT/US24/54646, filed 11/06/2024, assigned to the common owner and incorporated by reference herein, can enable individuals to define studies using natural language, making it far easier for individuals to design and run studies based on real-world evidence.

However, even when using the techniques in the above-referenced applications, users can still face significant limitations, as typically each study is defined and run individually. Running a comprehensive set of case studies is possible, but doing so would typically involve a significant amount of manual input on the part of users. Moreover, not all studies may produce useful results, resulting in wasted computing resources. Even if a user were to manually input and run a large number of studies, significant effort would be involved in understanding trends and/or commonalities across studies. Moreover, it may often simply be impractical for users to manually define a large number of studies. As an example, consider a simple scenario in which there are six diseases of interest, six drugs of interest, and six outcomes. Running every possible combination of disease, drug, and outcome would require 216 separate runs. If there are combinations of drugs, combinations of diseases, or combinations of outcomes, the number of runs needed to completely cover the problem space can grow rapidly.

Accordingly, there is a need for approaches that can enable users to more fully take advantage of the large datasets and computing power that are available. Described herein are approaches that can be used to automate the creation of batches of jobs to run multiple studies. In some implementations, the approaches herein improve efficiency by reusing or partially reusing the results of previous queries. In some implementations, the approaches herein dynamically scale processing power at runtime based on batch size or computing demands. In some implementations, the approaches herein segment batches for running in different environments. Some implementations analyze final results to provide a meta-analysis of the studies included in a batch. Some implementations automatically prioritize studies or tune study parameters based on the results of previously-run studies in a batch. These and other aspects of the approaches herein are described in more detail below.

The techniques described herein have the potential to significantly improve health. While there is an immense amount of data stored in electronic health records, claims records, and so forth, it has been difficult to fully take advantage of this information. Using the techniques described herein, researchers can more fully utilize the wealth of information available, potentially leading to significant, clinically applicable developments that have thus far gone undiscovered.

Running studies as batches rather than individually can offer various advantages. For example, batch processing can reduce the overhead associated with initiating and managing multiple individual jobs. Resource utilization can be improved. For example, a database connection can be reused, rather than having a separate, new connection for each individual job. Batch jobs can optimize query execution by combining multiple queries into a single transaction or by recycling queries across multiple jobs or even across different batches. For example, if multiple studies are to be run using a population of 45 to 65-year-old males, those individuals matching these criteria can be determined using one query, the results of which are shared across jobs in a batch. In some implementations, a cohort (e.g., 45 to 65-year-old males) can be further filtered before being analyzed. In some implementations, such additional filtering may not be used. For example, it may be faster to identify a cohort in an atomic manner rather than piecemeal. In some implementations, the same cohort may be used for multiple studies, in which case data for the cohort can be shared among multiple studies, each of which can perform different downstream processing. Batch jobs can be readily scaled across multiple processors or servers, enabling more efficient processing of large volumes of data. For example, different batch jobs can be run on different processors or servers. In some cases, there may be divisible aspects of a particular study, and the different divisible aspects can be run on different processors or servers.

Batch jobs present several challenges not typically encountered or that are lesser issues with individual jobs. For example, when executing batch jobs, it can be important to consider scheduling, monitoring, error handling, resource allocation, prioritization, and so forth.

In some implementations, a platform performs quality assurance while a batch is running. For example, as jobs within a batch complete, the platform can analyze the results of the jobs and determine whether or not the job produced statistically meaningful results (e.g., p value less than a threshold value, such as 0.05, 0.01, or 0.001). In some implementations, the platform prioritizes subsequent jobs based on this analysis. For example, if there are jobs in the queue but that have not yet run, the system can prioritize jobs that are more similar to studies with statistically significant results over those that are more similar to studies that did not produce statistically significant results. In some implementations, a user can configure the platform to automatically cancel jobs based on such analysis. For example, if a user has a limited budget for running studies, they may want to configure a batch to automatically cancel jobs that are unlikely to produce statistically significant results.

In some implementations, the platform employs one or more statistical methods depending on the nature of the data and the research question. For example, the platform may utilize chi-square tests for analyzing categorical outcomes, t-tests or ANOVA for comparing continuous outcomes across groups, and regression analysis (e.g., linear, logistic, or Cox proportional hazards) for modeling relationships between variables while controlling for confounders. In some implementations, the platform may employ survival analysis techniques such as Kaplan-Meier estimation and log-rank tests for time-to-event outcomes. In some implementations, the platform may utilize propensity score matching or inverse probability weighting to address selection bias in observational studies. In some implementations, the platform may employ meta-analysis techniques to combine results across multiple studies within a batch, providing more robust estimates of treatment effects.

In some implementations, the platform may automatically select appropriate statistical tests based on the characteristics of the data and the study design. For example, the platform may analyze the distribution of outcome variables to determine whether parametric or non-parametric tests are appropriate. The platform may assess sample sizes to determine whether asymptotic approximations are valid or whether exact tests are needed. The platform may identify potential violations of statistical assumptions (e.g., non-independence of observations, heteroscedasticity) and suggest appropriate remedies or alternative methods. In some implementations, the platform may provide sensitivity analyses that assess the robustness of findings to different statistical assumptions or methods.

In some implementations, the platform may provide various visualization and presentation options for study results. For example, the platform may generate forest plots that display effect sizes and confidence intervals across multiple studies in a batch. The platform may generate Kaplan-Meier curves for survival analyses, heat maps for displaying patterns across multiple variables, and interactive dashboards that enable users to explore results dynamically. In some implementations, the platform may generate natural language summaries of study results, translating statistical findings into plain language that is accessible to non-statisticians. In some implementations, the platform may generate reports in formats suitable for publication or regulatory submission, including tables, figures, and statistical summaries formatted according to relevant guidelines.

In some implementations, the platform may address the multiple comparisons problem that arises when running many studies in a batch. When numerous statistical tests are performed, the probability of obtaining false positive results increases. The platform may apply corrections for multiple comparisons, such as Bonferroni correction, Benjamini-Hochberg procedure for controlling false discovery rate, or other appropriate methods. In some implementations, the platform may report both unadjusted and adjusted p-values, enabling researchers to interpret results in the context of the multiple testing burden. The platform may provide guidance for the interpretation of results in the context of multiple comparisons, helping researchers distinguish between findings that are likely to be genuine and those that may be statistical artifacts.

In some implementations, the platform may assess statistical power and sample size adequacy for each study in a batch. For example, the platform may calculate post-hoc power for completed studies, indicating the probability that the study would have detected an effect of a given size if one existed. The platform may identify studies that are underpowered, flagging results that should be interpreted with caution due to insufficient sample size. In some implementations, the platform may perform prospective power calculations before executing jobs, enabling researchers to identify and potentially skip studies that are unlikely to have sufficient power to detect clinically meaningful effects, for example, because of small population size. The platform may suggest modifications to study parameters that would improve statistical power, such as broadening inclusion criteria or extending follow-up periods.

In some implementations, the platform may identify and handle outliers or influential observations that could affect study results. For example, the platform may apply statistical tests to identify observations that are unusually distant from the rest of the data. The platform may perform sensitivity analyses that exclude potential outliers, enabling researchers to assess whether conclusions are robust to the presence of extreme values. In some implementations, the platform may use robust statistical methods that are less sensitive to outliers, such as median-based measures or trimmed means. The platform may flag studies where outliers appear to have a substantial influence on results, prompting researchers to investigate further before drawing conclusions.

In some implementations, a platform is configured to automatically tune study parameters. For example, as jobs within a batch complete, the platform can analyze the results of the jobs and use this information to automatically tune parameters for subsequent studies. In some implementations, the tuning is done automatically, reducing the burden on researchers and helping ensure that appropriate tuning is carried out. For example, the platform can perform statistical analysis and determine which parameters are most impactful or the most statistically significant, and the platform can tune subsequent studies to vary those parameters. In some implementations, the platform utilizes only statistical analysis to tune parameters. In some implementations, the platform utilizes a machine learning model that is trained to identify relevant parameters, trends, etc., in the results of studies and to determine parameter values for subsequent studies. In some implementations, the platform generates new jobs. In some implementations, the platform modifies jobs that have not yet run. In some implementations, the platform is configured to automatically cancel certain jobs. For example, the platform can automatically cancel jobs that are unlikely to produce statistically significant results.

In some implementations, the platform may flag results that warrant further investigation based on various criteria. For example, the platform may highlight studies with unexpectedly large effect sizes, which may indicate either important findings or potential data quality issues. The platform may flag studies where results differ substantially from expectations based on prior knowledge or published literature. The platform may identify studies where subgroup analyses reveal heterogeneity in treatment effects, suggesting that certain patient populations may respond differently to interventions. In some implementations, the platform may use anomaly detection algorithms to identify unusual patterns in results that may merit closer examination.

In some implementations, the platform may compare study results to published literature or established benchmarks. For example, the platform may retrieve relevant published studies from medical literature databases and compare effect sizes, confidence intervals, or other metrics. The platform may flag results that are inconsistent with established evidence, prompting researchers to investigate potential explanations for the discrepancy. In some implementations, the platform may provide context for interpreting results by displaying how they compare to results from similar studies conducted on different populations or in different settings. This comparison can help researchers assess the generalizability of their findings and identify potential sources of heterogeneity.

In some implementations, the platform may generate hypotheses for follow-up studies based on the results of completed batches. For example, if a batch reveals that a treatment is particularly effective in a specific subgroup, the platform may suggest follow-up studies to further characterize this subgroup or to explore the mechanisms underlying the differential response. If a batch reveals unexpected associations between variables, the platform may suggest studies designed to investigate potential causal relationships. In some implementations, the platform may use machine learning to identify patterns across multiple study results that suggest promising avenues for further investigation. The platform may prioritize suggested follow-up studies based on factors such as potential clinical impact, feasibility, and alignment with the researcher's stated objectives.

In some implementations, a user can provide a cap on the number of studies to be run, a total budget, etc., and the platform can automatically tune parameters and the number of jobs to fit within the cap or budget while optimizing the study parameters so that the jobs that are run are more likely to produce meaningful results (e.g., as compared to randomly selecting jobs from a set of all possible jobs for a given set of parameters and parameter values).

As just one example of using a machine learning model to optimize jobs, the platform can be configured to utilize a clustering algorithm to cluster studies with similar results. The platform can then suggest that the user perform studies that are variations on those studies with significant results. In some implementations, the platform automatically configures subsequent jobs so that they are more likely to produce statistically significant results. Additionally or alternatively, the platform can utilize the machine learning model to identify variables that are least likely to produce statistically significant results when varied and may determine that such studies should not be run. In some implementations, the platform can modify a study definition. For example, if a researcher originally defined height as a variable with multiple groupings (e.g., less than five feet, five feet to six feet, more than six feet) but studies that have already run show no statistical significance to height, the platform can determine that height should be eliminated as a variable, and all heights should be considered together. As another example, if a researcher originally defined a study to include aspirin dosage as a variable with a small number of dosage bins, and results of studies already run show that dosage is a significant driver of outcome, the platform can automatically increase the number of bins to consider finer dosing ranges, for example dividing the range 100 mg – 500 mg into 100 mg – 200 mg, 201 mg – 300 mg, 301 mg – 400 mg, and 401 mg – 500 mg. In some implementations, the platform is configured to confirm such changes with a user before proceeding with the changes. In some implementations, changes can be made without user input, or a user can provide permission ahead of time to modify study parameters.

Designing a multi-variable, multi-way set of studies can be challenging. Manually crafting queries for each study to be run can be both time-consuming and error-prone. Thus, there is a need for approaches that can simplify the process of creating a potentially large number of jobs (individual studies) in a batch. As described herein, a platform can provide a straightforward user interface that can be used to define multiple studies without manually defining each study. For example, a single base study can be defined, along with a set of variables to be substituted into the base study to conduct each individual study. The base study can be a query (e.g., a TQL query) that is modified to accept variable values in defined places. For example, instead of specifying an age range in the base query, the base query can specify that the age range is a variable, and particular age ranges can be selected for individual queries from a set of age ranges defined or selected by the user or automatically suggested by the platform.

In some implementations, the platform may provide audit trail and reproducibility features to support regulatory compliance and scientific rigor. For example, the platform may maintain detailed logs of all actions taken during batch definition, execution, and analysis, enabling researchers to demonstrate exactly how results were obtained. The platform may capture the versions of software components, data sources, configuration parameters, etc., used in each batch, enabling results to be reproduced at a later time. In some implementations, the platform may generate documentation suitable for inclusion in regulatory submissions, clinical study reports, or scientific publications. The platform may support electronic signatures and approval workflows, enabling organizations to implement appropriate governance controls over study execution.

In some implementations, the platform may support collaboration among multiple users working on batch definitions. For example, multiple researchers may simultaneously or sequentially edit a batch definition, with the platform managing concurrent access and resolving conflicts. The platform may provide commenting and annotation features, enabling team members to discuss design decisions and document rationale. The platform may track contributions from different users, maintaining an audit trail of who made what changes and when. In some implementations, the platform may support role-based access control, enabling organizations to define who can view, edit, execute, or approve batches.

In some implementations, the platform may provide version control capabilities for batch definitions. For example, the platform may maintain a history of changes to batch definitions, enabling researchers to review how a batch has evolved over time. The platform may enable researchers to revert to previous versions of a batch definition if needed. The platform may support branching and merging, enabling researchers to explore alternative study designs without affecting the main batch definition. In some implementations, the platform may enable researchers to clone existing batches, creating copies that can be modified for new purposes while preserving the original batch intact.

In some implementations, the platform may enable sharing of batches and results across users or organizations. For example, researchers may publish batch definitions to a shared repository, enabling others to reuse or adapt proven study designs. The platform may support export and import of batch definitions in standard formats, facilitating collaboration across different instances of the platform. In some implementations, the platform may provide mechanisms for sharing results while protecting sensitive information, such as by providing aggregate statistics without exposing individual-level data. The platform may support the creation of templates based on successful batches, enabling organizations to standardize their approach to common types of studies.

In some implementations, the platform may integrate with external tools commonly used in medical research workflows. For example, the platform may export results in formats compatible with statistical packages such as R, SAS, or Stata, enabling researchers to perform additional analyses using familiar tools. The platform may integrate with reference management software, facilitating the incorporation of study results into manuscripts and publications. The platform may integrate with regulatory submission systems or support the generation of regulatory forms or the filling of existing regulatory forms, streamlining the process of preparing study results for submission to regulatory authorities. In some implementations, the platform may provide APIs that enable programmatic access to batch definitions and results, supporting integration with custom tools and automated workflows.

In some implementations, the platform utilizes machine learning-based job prioritization to optimize the execution of jobs within a batch. For example, the platform may train a machine learning model on historical data about job execution times, resource requirements, and result quality. The model may predict which jobs are most likely to complete quickly, which jobs are most likely to produce statistically significant results, which jobs are most likely to be of interest to the user, etc. Using these predictions, the platform can prioritize jobs to improve the value delivered to the user within available time and resource constraints. In some implementations, the model is continuously updated based on the results of jobs, enabling the platform to adapt its prioritization strategy in real time. In some implementations, the model is updated periodically or as requested, which can enable model improvement while helping ensure that outputs are consistent from run to run.

In some implementations, the platform utilizes federated learning approaches, in which analysis occurs at data source locations (e.g., a hospital system can run analysis on its own data) rather than centralizing data, or in which only limited data is shared for processing jobs, which can help limit potential data sharing risks. In such implementations where jobs are run at data source locations, the platform may distribute analysis tasks to computing resources located at or near the data sources, and a central location can aggregate results to produce final outputs. This approach can address privacy concerns by ensuring that raw patient data never leaves the institution where it was collected. It can also reduce network bandwidth requirements by transmitting only summary statistics or other outputs or intermediate results rather than raw data. In some implementations, the platform utilizes secure multi-party computation, differential privacy techniques, etc., to further protect patient privacy while enabling meaningful analysis across distributed data sources.

In some implementations, the platform utilizes caching and result reuse strategies to improve efficiency when similar queries are executed. For example, if multiple jobs in a batch require data for the same patient population, the platform may retrieve that data once and cache it for reuse by subsequent jobs. If a job is similar to a previously executed job, the platform may reuse relevant portions of the previous results rather than recomputing them from scratch. In some implementations, the platform maintains a cache of intermediate results (e.g., filtered patient populations, computed covariates) that can be reused across jobs. In some implementations, the platform utilizes content-addressable storage or other storage techniques to help identify opportunities for result reuse based on the semantic content of queries rather than their syntactic form.

In some implementations, the platform dynamically selects between parallel and sequential execution strategies based on resource availability, job characteristics, and/or other factors. For example, when ample computing resources are available, the platform can execute multiple jobs in parallel to reduce run time. When resources are constrained, the platform may execute jobs sequentially to avoid resource contention. In some cases, there can be dependencies between jobs, and such jobs can be executed sequentially. In some implementations, the platform may utilize a hybrid approach, executing some jobs in parallel while queuing others for sequential execution. The platform may consider factors such as job priority, estimated execution time, resource requirements, and dependencies between jobs when making scheduling decisions. In some implementations, the platform dynamically scales computing resources (e.g., by provisioning additional cloud computing instances) based on the size and urgency of the batch. In some implementations, a user specifies the urgency. For example, the platform can implement a tiered fee structure in which users can opt to pay more to prioritize jobs.

### Illustrative Examples

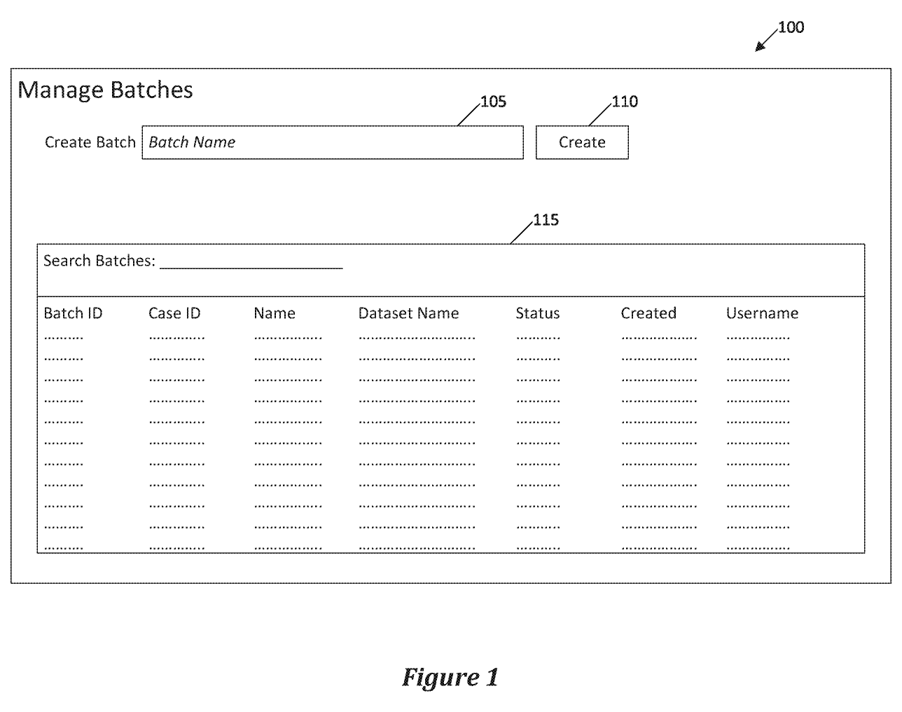

FIG. 1 is a drawing that illustrates an example user interface 100 for batch management according to some implementations. In FIG. 1, a batch management user interface 100 provides functionality for a user to create a new batch, for example using batch name input box 105 and create batch button 110. The batch management user interface 100 of FIG. 1 enables users to search existing batches or view existing batches using batch browser 115. For example, a user can select a batch from the list of batches (which can be filtered in some implementations) in order to view the batch or carry out operations on the batch.

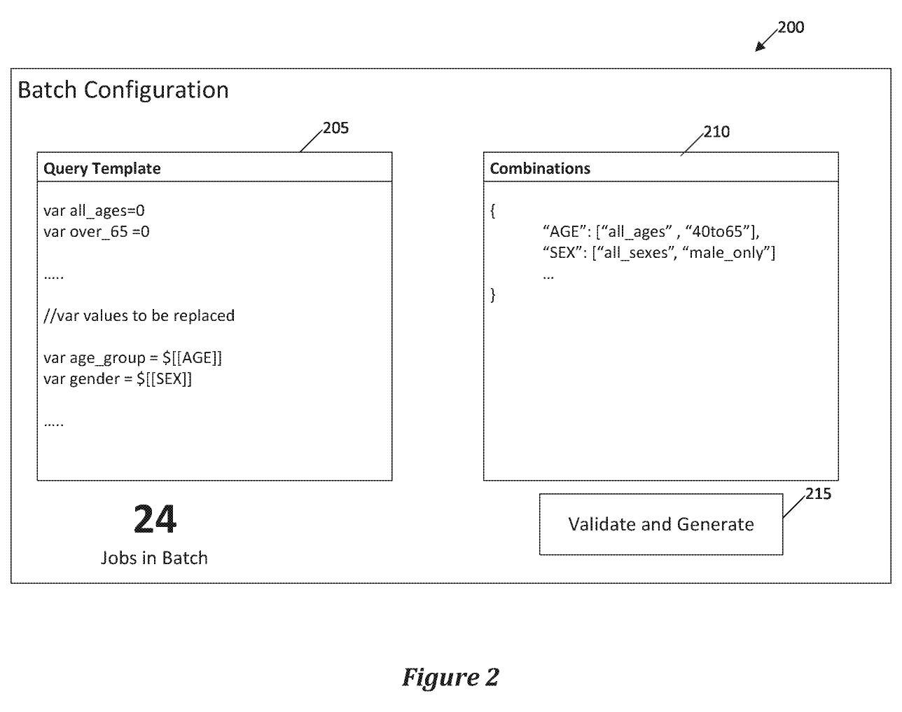

FIG. 2 is a drawing that illustrates an example batch configuration user interface 200 according to some implementations. In FIG. 2, the batch configuration user interface 200 includes query template field 205 and combination field 210. The query template field 205 can specify syntax for a query to be executed. The query can be parameterized such that certain variables can be replaced with different values. For example, in FIG. 2, age_group and gender are set as variable parameters. The combinations field 210 enables users to define specific combinations of variables to be used in generating batch jobs. For example, in FIG. 2, the “AGE” parameter is replaced in the query template with “all_ages” and “40to65” and the “SEX” parameter is replaced with “all_sexes” and “male_only.” The batch configuration user interface indicates the total number of jobs in the batch. The total number of jobs can be combined as the product of the number of different values for each parameter that varies from job to job. In FIG. 2, 24 total jobs are indicated (e.g., there are more variable parameters than are shown in the example interface of FIG. 2). The batch configuration interface includes a button 215 or other user interface element to enable a user to validate the query and combinations and generate individual jobs. The validation can include, for example, checking that the query adheres to a defined syntax, that the combinations adhere to a defined syntax (e.g., JavaScript Object Notation (JSON)). If there are errors, the batch configuration user interface can provide an error message to the user. In some implementations, the error message indicates a location or other description of the error.

In some implementations, the query template supports various types of parameters beyond simple value substitution. For example, the query template may support parameters for treatment dosages, enabling researchers to explore dose-response relationships by varying dosage thresholds or ranges across jobs. The query template may support parameters for time windows, enabling researchers to explore how the timing of treatments or outcomes affects study results. The query template may support parameters for outcome definitions, enabling researchers to explore how different definitions of success or failure affect conclusions. In some implementations, the query template may support nested or hierarchical parameters, enabling researchers to define complex study designs with multiple levels of variation. which can simplify the study definition process and can limit the number of studies performed. For example, nesting can be used to explore a certain group more deeply while not doing the same exploration across all groups.

In some implementations, the platform may validate parameter combinations for clinical meaningfulness before generating jobs. For example, the platform may check that specified age ranges are clinically plausible, that treatment dosages fall within approved ranges, or that outcome time windows are appropriate for the condition under study. The platform may flag potentially problematic parameter combinations, such as combinations that would result in very small sample sizes or combinations that represent clinically implausible scenarios (e.g., dosages too high or combinations of medications that would have serious side effects). In some implementations, the platform may suggest alternative parameter values based on clinical guidelines, published literature, or patterns observed in the underlying data. This validation can help researchers avoid wasting computational resources on studies that are unlikely to yield meaningful results.

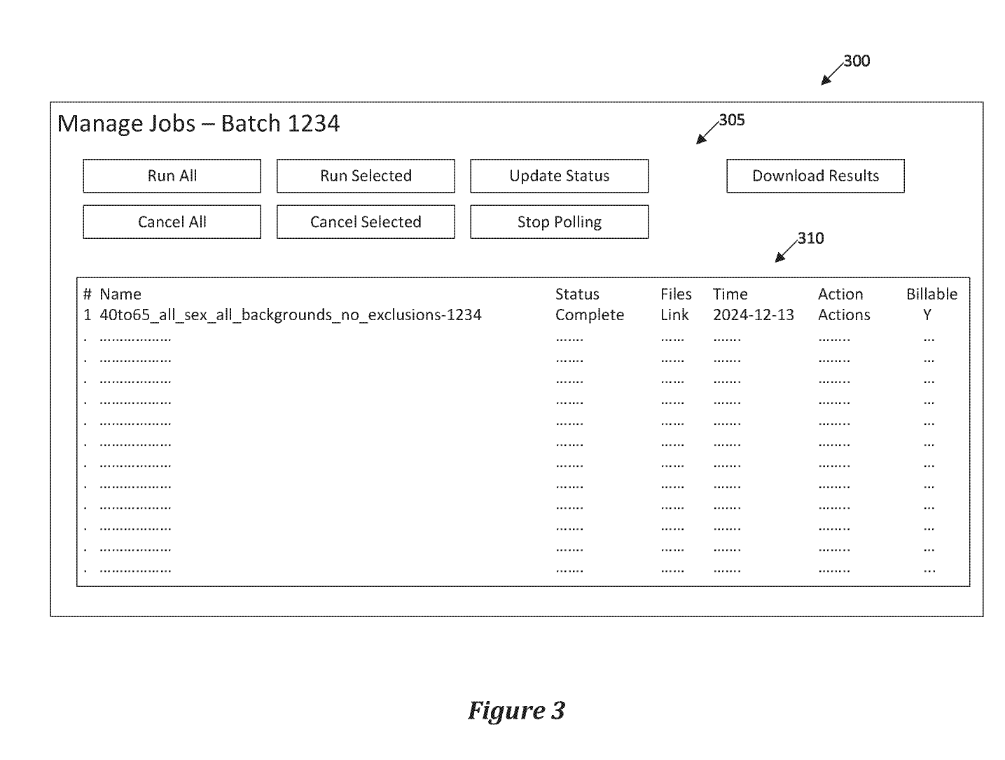

FIG. 3 is a drawing that illustrates an example user interface for managing jobs in a batch according to some implementations. As described herein, a batch can include a plurality of jobs. The jobs can run in parallel or in series. The jobs can run on one system or on multiple computer systems. The user interface 300 provides various functionality for monitoring and interacting with jobs within a batch. The user interface 300 can provide a list 310 of jobs within a batch (in the example of FIG. 3, the jobs included in the batch “1234”). Jobs within a batch can have descriptive names based on variable parameter values. For example, job #1 in FIG. 3 includes subjects aged 40 to 65 of all genders and backgrounds without any exclusions. In some implementations, the job name includes the batch name or another indication of which batch the job belongs to. For example, “1234” is appended to the end of the name of job #1 in the example of FIG. 3, indicating that the job is part of batch 1234. The user interface 300 can include various information about each job. For example, in the listing of jobs, the status of each job can be listed, as well as a link to files associated with the job, a time associated with the job (e.g., a submission time, start time, end time, etc.), an indication of whether or not the job is billable (e.g., some jobs may be billable while others may be done for free or as part of initial setup or test runs that are not billed to a client). The listing can include various combinations of information, and in some implementations, can be customized by users. In some implementations, the listing enables users to access various actions, such as forcing a job to start, terminating a job, renaming a job, editing a job, and so forth. The user interface 300 can include various buttons and/or other user interface elements. For example, in FIG. 3, the user interface 300 includes buttons 305 to run all the jobs in the batch, cancel all the jobs in the batch, run selected jobs in the batch (e.g., jobs selected in the listing), and cancel selected jobs. In FIG. 3, the user interface 300 includes a button to update the status of the jobs. In some implementations, the status is automatically updated, either continuously (e.g., whenever there is a change in the status of a job) or periodically (e.g., refreshed every one minute, ten minutes, one hour, etc.). In some implementations, the status can be refreshed manually in response to a user request to update the status of the jobs. In FIG. 3, the user interface 300 includes a button to stop polling. This can cause a system to stop updating the status of the jobs. This can be desirable in some circumstances, for example, to reduce load on servers or to free up resources that would be used for monitoring so that they can be used for other tasks, such as retrieving data, analyzing data, generating reports, and so forth. The user interface 300 can include a button to download all the available results for all the jobs in the batch, although, as described herein, the user interface 300 can also provide functionality for downloading or otherwise accessing the results of individual jobs in the batch.

In some implementations, the platform may provide functionality for pausing, resuming, or restarting jobs. For example, a user may pause a batch to free up computing resources for higher-priority work, then resume the batch later when resources become available. As another example, a user may pause a batch to review the results of individual jobs that have completed before continuing with executing the batch. The platform may automatically checkpoint job progress, enabling jobs to be resumed from the point of interruption rather than restarting from the beginning. In some implementations, the platform may automatically pause jobs that encounter transient errors (such as temporary network connectivity issues) and retry them after a configurable delay. The platform may provide options for restarting failed jobs with modified parameters, enabling researchers to address issues identified during initial execution attempts.

In some implementations, the platform provides various mechanisms for handling job failures and retries. For example, the platform may automatically retry jobs that fail due to transient errors, such as network timeouts or temporary resource unavailability. The platform may implement exponential backoff strategies, waiting progressively longer between retry attempts to avoid overwhelming stressed systems. The platform may distinguish between recoverable errors (which may be addressed through retries) and non-recoverable errors (which require user intervention), handling each appropriately. In some implementations, the platform may quarantine jobs that repeatedly fail, preventing them from consuming resources while enabling the rest of the batch to proceed.

In some implementations, the platform may provide various notification mechanisms to keep users informed about job status. For example, the platform may send email notifications when jobs complete, fail, or require attention. The platform may provide push notifications to mobile devices, enabling users to monitor batch progress while away from their workstations. The platform may integrate with messaging platforms such as Slack or Microsoft Teams, posting status updates to designated channels. In some implementations, the platform may provide configurable notification thresholds, enabling users to receive notifications only for significant events (such as batch completion or critical errors) rather than for every job status change.

In some implementations, the platform may estimate job completion times based on historical data and current system conditions. For example, the platform may analyze the execution times of similar jobs from previous batches to predict how long current jobs are likely to take. The platform may adjust estimates based on current system load, accounting for the fact that jobs may take longer when computing resources are heavily utilized. The platform may provide confidence intervals or ranges for completion time estimates, reflecting uncertainty in the predictions. In some implementations, the platform may update completion time estimates as jobs progress, providing increasingly accurate predictions as more information becomes available.

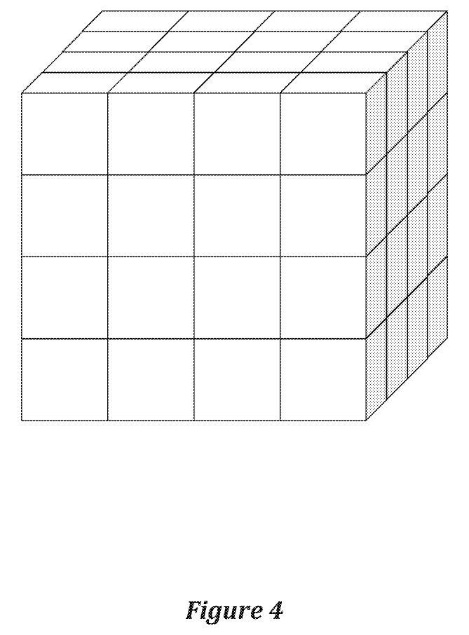

FIG. 4 is a drawing that schematically illustrates multivariable jobs. In FIG. 4, there are three parameters that are changed, each with four possible values, resulting in a total of 4 x 4 x 4 = 64 combinations of variables, and thus 64 separate jobs to run if all combinations of values are to be tested. As the number of variables grows or the number of possible values grows, the number of jobs can easily grow into hundreds, thousands, or even more jobs. For example, if there are still three parameters but each can have 8 values, then the number of jobs is 8 x 8 x 8 = 512 jobs.

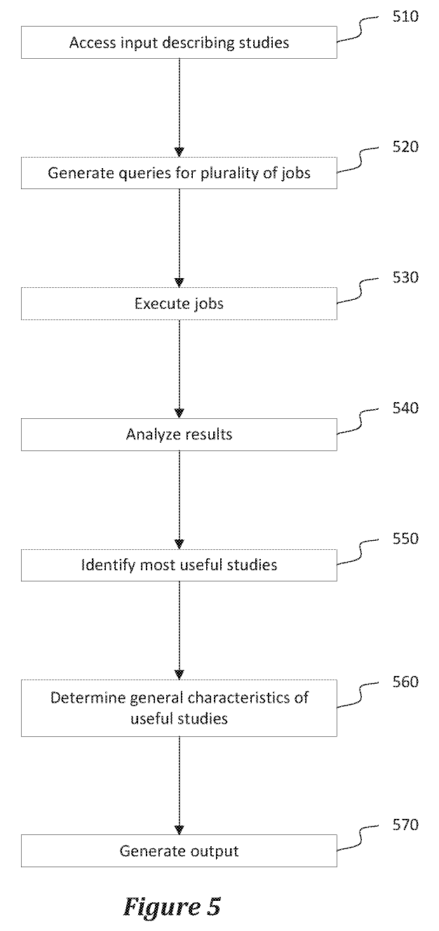

FIG. 5 is a flowchart that illustrates an example batch processing process according to some implementations. At operation 510, a system can access input describing studies to be carried out. For example, the input can be a natural language input that describes the study to be carried out and the parameters to be varied. In some cases, a user may specify values for the parameters. In other cases, a user may provide a more general description, such as “all medications for treating high blood pressure,” and the system can utilize an information source such as a phenotype library to identify the relevant medications. In some implementations, a user submits actual query language, JSON to describe the variables and their parameters, and so forth, rather than using natural language to describe the studies to be performed. In the case that a user uses a natural language description, the system can provide the natural language description to a large language model to determine the parameters and values for the studies. The system can provide this information to the user for confirmation.

At operation 520, the system can generate queries for a plurality of jobs. For example, if the study includes one variable parameter with five possible values, the system can generate five jobs and their corresponding queries (e.g., one job for each possible value of the variable parameter). At operation 530, the system can execute the jobs. As described herein, the jobs can be executed on one system or multiple systems. In some implementations, the system monitors the status of each job, for example, to determine if individual jobs are queued, running, in an error state, canceled, completed, etc.

At operation 540, the system can perform statistical analysis of the results of each job. At operation 550, the system can, based on the analysis, identify the most useful studies. For example, the most useful studies can be those studies with p values below a threshold value (e.g., p < 0.05, p < 0.01, p < 0.001, etc.). Studies with p values above the threshold may be less useful, as the likelihood of getting such a result even if the null hypothesis is true may be too high.

In some cases, studies that are most useful (e.g., those with low p values) may have common characteristics. At operation 560, the system can determine general characteristics of the most useful studies. As an example, the system may determine that studies that are restricted to older populations are most useful, studies that are restricted to only men or only women are most useful, studies involving a certain drug or treatment are most useful, and so forth. At operation 570, the system can generate an output that indicates, for example, summary statistics of the individual studies, identification of which studies were determined to be most useful, information about the general characteristics of the most useful studies, and so forth.

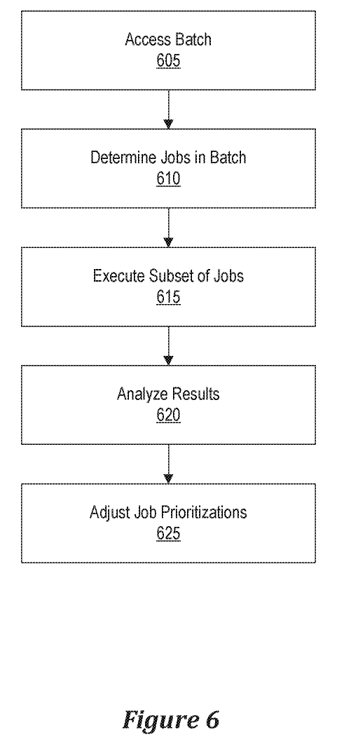

FIG. 6 is a flowchart that illustrates an example process for optimizing jobs in a batch according to some implementations. At operation 605, a platform can access a batch. At operation 610, the platform can determine the jobs in the batch. At operation 615, the platform can execute a subset of jobs in the batch. In some implementations, the subset of jobs is selected randomly, in a first-in-first-out (FIFO) order, in a first-in-last-out (FILO) order, etc. As individual jobs within the batch complete, the platform can analyze the results at operation 620. The platform can use the analysis to determine which jobs produced actionable results, for example, to identify which jobs showed statistically significant differences in outcomes based on treatment, demographic factors, co-morbidities, etc. At operation 625, the platform can adjust the prioritization (e.g., execution order) of remaining jobs, for example, so that jobs that are more similar to those that produced significant results are executed before other jobs.

In FIG. 6, the platform alters the prioritization of jobs based on the statistical significance of jobs that have already executed, but does not tune the parameters for subsequent jobs. In some cases, it may be desirable to tune parameters for subsequent jobs, for example, as described herein (e.g., by doing more and or less fine-grained variations of a parameter, adding a new parameter, or eliminating a parameter).

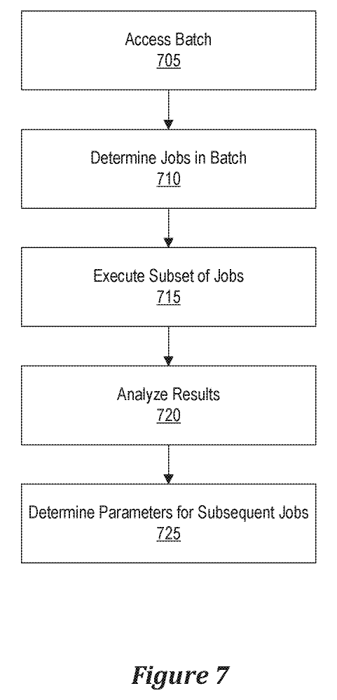

FIG. 7 is a flow chart that illustrates another example process for optimizing jobs in a batch according to some implementations. At operation 705, a platform can access a batch. At operation 710, the platform can determine the jobs in the batch. At operation 715, the platform can execute a subset of jobs in the batch. In some implementations, the subset of jobs is selected randomly, in a first-in-first-out (FIFO) order, in a first-in-last-out (FILO) order, etc. As individual jobs within the batch complete, the platform can analyze the results at operation 720. The platform can use the analysis to determine which jobs produced actionable results, for example, to identify which jobs showed statistically significant differences in outcomes based on treatment, demographic factors, co-morbidities, etc. At operation 725, the platform can determine parameters and/or parameter values for subsequent jobs. For example, the platform can identify parameters and/or parameter values that are most likely to produce statistically significant results based on the analysis at operation 720 and can modify subsequent jobs and/or generate additional jobs to further explore such parameters.

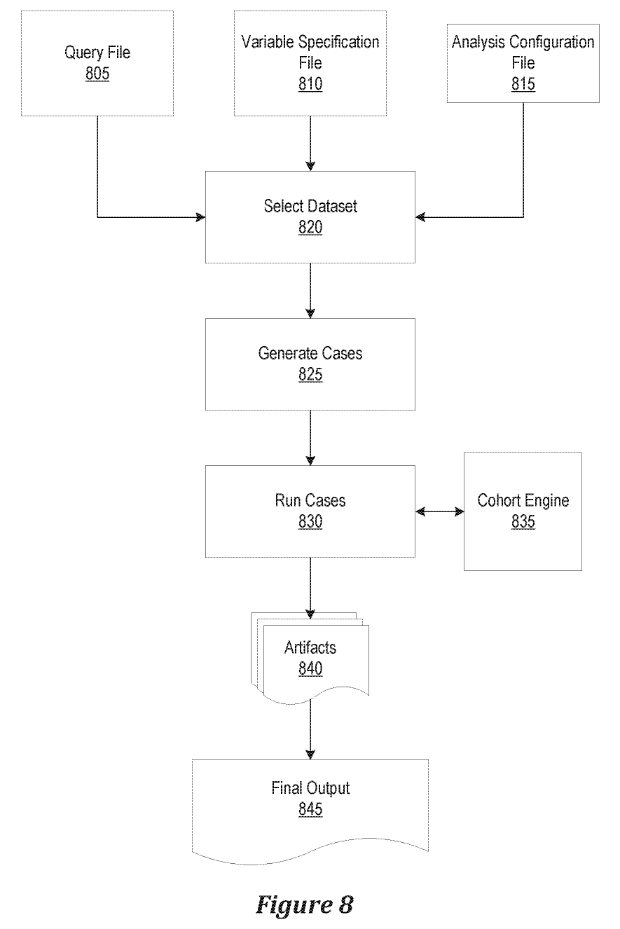

FIG. 8 is a flowchart that illustrates an example batch processing implementation. A system can access a query file 805, a variable specification file 810, and an analysis configuration file 815. The query file 805 can specify a base query, while the variable specification file 810 can specify how variable values are to be changed for different batches, and the analysis configuration file 815 can specify what analyses to carry out.

At operation 820, the system can select a dataset, either as specified by a user or based on the information in the query file 805 and/or the variable specification file 810. At operation 825, the system can generate cases. Each case can be a job within a batch of jobs. At operation 830, the system can run the cases, which can include calling a cohort engine 835 to determine which subjects to include in each case. The system can output artifacts 840 for each case. The system can analyze and combine the artifacts 840 to generate a final output 845, which can be, for example, a report that summarizes the findings from the batch.

### Example Implementations

Implementation 1. A method for batch processing of medical studies, comprising: receiving, by a computing system, input describing a plurality of medical studies to be carried out, wherein the input includes a query template and a set of variable parameters with corresponding values; determining, by the computing system, a plurality of parameters for a plurality of jobs based on the input; generating, by the computing system, the plurality of jobs based on the query template and combinations of the variable parameters, wherein each job corresponds to a distinct combination of parameter values; executing, by the computing system, a subset of the plurality of jobs to produce results for each executed job; performing, by the computing system, statistical analysis on the results of the executed jobs to identify jobs that produced statistically significant results; based on the statistical analysis, determining, by the computing system, parameters or parameter values for subsequent jobs in the plurality of jobs; and modifying at least one of the subsequent jobs or generating additional jobs to further explore parameters associated with the statistically significant results.

Implementation 2. The method of implementation 1, wherein the input comprises a natural language description of the plurality of medical studies, and wherein the method further comprises processing the natural language description using a large language model to determine the query template and the set of variable parameters.

Implementation 3. The method of implementation 1, wherein the statistically significant results comprise results having a p value below a threshold value.

Implementation 4. The method of implementation 3, wherein the threshold value is one of 0.05, 0.01, or 0.001.

Implementation 5. The method of implementation 1, wherein modifying at least one of the subsequent jobs comprises: increasing a number of parameter value bins for a parameter determined to be a significant driver of outcome; or eliminating a parameter determined to have statistical significance below a threshold value.

Implementation 6. The method of implementation 1, further comprising adjusting a prioritization of remaining jobs in the plurality of jobs based on the statistical analysis, wherein jobs that are more similar to the jobs that produced statistically significant results are prioritized for execution before other jobs.

Implementation 7. The method of implementation 1, further comprising: utilizing a machine learning model to cluster studies with similar results; and based on the clustering, identifying variables that are most likely to produce statistically significant results when varied.

Implementation 8. The method of implementation 7, wherein the machine learning model comprises a clustering algorithm trained to identify relevant parameters or trends in results of the executed jobs.

Implementation 9. The method of implementation 1, further comprising: receiving a cap on a number of studies to be run or a total budget; and automatically tuning the parameters and a number of jobs to fit within the cap or the total budget while optimizing study parameters so that jobs that are run are more likely to produce statistically significant results.

Implementation 10. The method of implementation 1, further comprising: generating an output indicating summary statistics of the executed jobs; identifying which jobs were determined to produce statistically significant results; and determining general characteristics of the jobs that produced statistically significant results.

Implementation 11. A system for batch processing of medical studies, comprising: one or more processors; and a memory storing instructions that, when executed by the one or more processors, cause the system to: receive input describing a plurality of medical studies to be carried out, wherein the input includes a query template specifying syntax for queries to be executed and a combinations specification defining variable parameters and corresponding values for the variable parameters; generate a plurality of jobs based on the query template and the combinations specification, wherein each job corresponds to a distinct combination of values for the variable parameters; execute a subset of the plurality of jobs against one or more data sources containing medical data to produce results for each executed job; analyze the results of the executed jobs to determine which jobs produced statistically significant results based on a threshold p-value; and adjust prioritization of remaining jobs in the plurality of jobs based on the analysis, wherein jobs that are more similar to jobs that produced statistically significant results are prioritized for execution before jobs that are less similar to jobs that produced statistically significant results.

Implementation 12. The system of implementation 11, wherein the instructions further cause the system to modify at least one of the remaining jobs based on the analysis by increasing a number of parameter value bins for a parameter determined to be a significant driver of outcome.

Implementation 13. The system of implementation 11, wherein the instructions further cause the system to modify at least one of the remaining jobs based on the analysis by eliminating a parameter determined to have no statistical significance across the executed jobs.

Implementation 14. The system of implementation 11, wherein the instructions further cause the system to generate additional jobs to further explore parameters associated with the statistically significant results.

Implementation 15. The system of implementation 14, wherein generating additional jobs comprises creating jobs with finer-grained variations of parameter values for parameters identified as significant drivers of outcome.

Implementation 16. The system of implementation 11, wherein the instructions further cause the system to: receive a cap on a number of jobs to be executed or a total budget; and automatically tune the variable parameters and a number of jobs to fit within the cap or the total budget while optimizing study parameters so that jobs that are executed are more likely to produce statistically significant results compared to randomly selecting jobs.

Implementation 17. The system of implementation 11, wherein the instructions further cause the system to utilize a machine learning model to identify patterns across results of the executed jobs, wherein the machine learning model comprises a clustering algorithm configured to cluster studies with similar results.

Implementation 18. The system of implementation 17, wherein the instructions further cause the system to utilize the machine learning model to identify variables that are least likely to produce statistically significant results when varied and to determine that jobs involving such variables should not be executed.

Implementation 19. The system of implementation 11, wherein the one or more data sources containing medical data comprise at least one of electronic health records, claims data, pharmacy data, or public health tracking databases.

Implementation 20. The system of implementation 11, wherein the instructions further cause the system to generate an output indicating summary statistics of the executed jobs, identification of which jobs produced statistically significant results, and general characteristics of the jobs that produced statistically significant results.

### Computer System

In some implementations, the platform may segment batches for execution in different computing environments based on data locality, security requirements, or other constraints. For example, jobs that require access to data from a particular institution may be executed in a computing environment that has access to that institution's data. Jobs that involve particularly sensitive data may be executed in a secure enclave or other protected environment. Jobs that require specialized hardware (e.g., GPUs for machine learning tasks) may be routed to computing environments that have such hardware available. In some implementations, the platform may automatically determine the optimal execution environment for each job based on its requirements and the available resources.

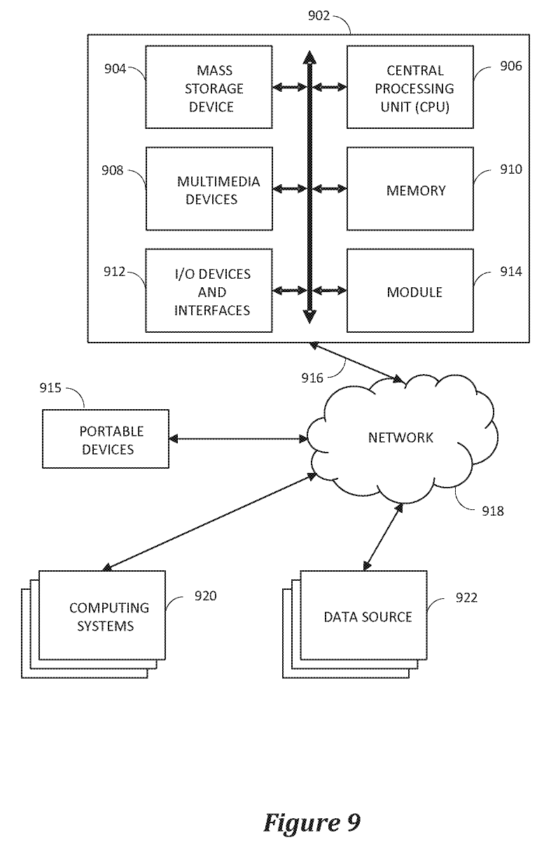

FIG. 9 is a block diagram 900 depicting an embodiment of a computer hardware system 902 configured to run software for implementing one or more of the systems and methods described herein. The example computer system 902 is in communication with one or more computing systems 920 and/or one or more data sources 922 via one or more networks 918. While FIG. 9 illustrates an embodiment of a computing system 902, it is recognized that the functionality provided for in the components and modules of computer system 902 may be combined into fewer components and modules, or further separated into additional components and modules.

The computer system 902 can comprise a module 914 that carries out the functions, methods, acts, and/or processes described herein. The module 914 is executed on the computer system 902 by a central processing unit 906 discussed further below.

In general, the word "module," as used herein, refers to logic embodied in hardware or firmware or to a collection of software instructions, having entry and exit points. Modules are written in a programming language, such as Java, Python, Rust, Ruby, PERL, BASIC, Lua, C, C++, C#, JavaScript, or the like. Software modules may be compiled or linked into an executable program, embodied in a dynamic link library, etc. In some implementations, modules are written in an interpreted language and provided as code. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 902 includes one or more processing units (CPU) 906, which may comprise a microprocessor. The computer system 902 further includes a physical memory 910, such as random-access memory (RAM) for temporary storage of information, a read only memory (ROM) for permanent storage of information, and a mass storage device 904, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 902 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 902 includes one or more input/output (I/O) devices and interfaces 912, such as a keyboard, mouse, touch pad, and printer. The I/O devices and interfaces 912 can include one or more display devices, such as a monitor, which allows the visual presentation of data to a user. More particularly, a display device provides for the presentation of GUIs as application software data, and multi-media presentations, for example. The I/O devices and interfaces 912 can also provide a communications interface to various external devices. The computer system 902 may comprise one or more multi-media devices 908, such as speakers, video cards, graphics accelerators, and microphones, for example.

The computer system 902 may run on a variety of computing devices, such as a server, a Windows server, a Structure Query Language server, a Unix Server, a personal computer, a laptop computer, and so forth. In other embodiments, the computer system 902 may run on a cluster computer system, a mainframe computer system and/or other computing system suitable for controlling and/or communicating with large databases, performing high volume transaction processing, and generating reports from large databases. The computing system 902 is generally controlled and coordinated by an operating system software, such as z/OS, Windows, Linux, UNIX, BSD, SunOS, Solaris, MacOS, or other compatible operating systems, including proprietary operating systems. Operating systems control and schedule computer processes for execution, perform memory management, provide file system, networking, and I/O services, and provide a user interface, such as a graphical user interface (GUI), among other things.

The computer system 902 illustrated in FIG. 9 is coupled to a network 918, such as a LAN, WAN, or the Internet via a communication link 916 (wired, wireless, or a combination thereof). Network 918 communicates with various computing devices and/or other electronic devices, such as portable devices 915. Network 918 is communicating with one or more computing systems 920 and one or more data sources 922. The module 914 may access or may be accessed by computing systems 920 and/or data sources 922 through a web-enabled user access point. Connections may be a direct physical connection, a virtual connection, and other connection type. The web-enabled user access point may comprise a browser module that uses text, graphics, audio, video, and other media to present data and to allow interaction with data via the network 918.

Access to the module 914 of the computer system 902 by computing systems 920 and/or by data sources 922 may be through a web-enabled user access point such as the computing systems' 920 or data source's 922 personal computer, cellular phone, smartphone, laptop, tablet computer, e-reader device, audio player, or another device capable of connecting to the network 918. Such a device may have a browser module that is implemented as a module that uses text, graphics, audio, video, and other media to present data and to allow interaction with data via the network 918.

The output module may be implemented as a combination of an all-points addressable display such as a cathode ray tube (CRT), a liquid crystal display (LCD), a plasma display, or other types and/or combinations of displays. The output module may be implemented to communicate with interfaces 912 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some implementations, the system 902 may comprise a physical or logical connection established between a remote microprocessor and a mainframe host computer for the express purpose of uploading, downloading, or viewing interactive data and databases on-line in real time. The remote microprocessor may be operated by an entity operating the computer system 902, including the client server systems or the main server system, an/or may be operated by one or more of the data sources 922 and/or one or more of the computing systems 920. In some implementations, terminal emulation software may be used on the microprocessor for participating in the micro-mainframe link.

In some implementations, computing systems 920 who are internal to an entity operating the computer system 902 may access the module 914 internally as an application or process run by the CPU 906.

In some implementations, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name, and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a website). The cookie data can be encrypted to provide security for the creator. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 902 may include one or more internal and/or external data sources (for example, data sources 922). In some implementations, one or more of the data repositories and the data sources described above may be implemented using a relational database, such as DB2, Sybase, Oracle, CodeBase, and Microsoft® SQL Server as well as other types of databases such as a flat-file database, an entity relationship database, and object-oriented database, and/or a record-based database.

The computer system 902 may also access one or more databases 922. The databases 922 may be stored in a database or data repository. The computer system 902 may access the one or more databases 922 through a network 918 or may directly access the database or data repository through I/O devices and interfaces 912. The data repository storing the one or more databases 922 may reside within the computer system 902.

### Remarks

The terms “example,” “embodiment,” and “implementation” are used interchangeably. For example, references to “one example” or “an example” in the disclosure can be, but are not necessarily, references to the same implementation; and such references mean at least one of the implementations. The appearances of the phrase “in one example” are not necessarily all referring to the same example, nor are separate or alternative examples mutually exclusive of other examples. A feature, structure, or characteristic described in connection with an example can be included in another example of the disclosure. Moreover, various features are described that can be exhibited by some examples and not by others. Similarly, various requirements are described that can be requirements for some examples but not for other examples.

The terminology used herein should be interpreted in its broadest reasonable manner, even though it is being used in conjunction with certain specific examples. The terms used in the disclosure generally have their ordinary meanings in the relevant technical art, within the context of the disclosure, and in the specific context where each term is used. A recital of alternative language or synonyms does not exclude the use of other synonyms. Special significance should not be placed upon whether or not a term is elaborated or discussed herein. The use of highlighting has no influence on the scope and meaning of a term. Further, it will be appreciated that the same thing can be said in more than one way.

Unless the context clearly requires otherwise, throughout the description and the claims, the words “comprise,” “comprising,” and the like are to be construed in an inclusive sense, as opposed to an exclusive or exhaustive sense—that is to say, in the sense of “including, but not limited to.” As used herein, the terms “connected,” “coupled,” or any variants thereof mean any connection or coupling, either direct or indirect, between two or more elements; the coupling or connection between the elements can be physical, logical, or a combination thereof. Additionally, the words “herein,” “above,” “below,” and words of similar import can refer to this application as a whole and not to any particular portions of this application. Where context permits, words in the Detailed Description above using the singular or plural number may also include the plural or singular number, respectively. The word “or” in reference to a list of two or more items covers all of the following interpretations of the word: any of the items in the list, all of the items in the list, and any combination of the items in the list. The term “module” refers broadly to software components, firmware components, and/or hardware components.

While specific examples of technology are described above for illustrative purposes, various equivalent modifications are possible within the scope of the present disclosure, as those skilled in the relevant art will recognize. For example, while processes or blocks are presented in a given order, alternative implementations can perform routines having steps, or employ systems having blocks, in a different order, and some processes or blocks may be deleted, moved, added, subdivided, combined, and/or modified to provide alternative or sub-combinations. Each of these processes or blocks can be implemented in a variety of different ways. Also, while processes or blocks are at times shown as being performed in series, these processes or blocks can instead be performed or implemented in parallel, or can be performed at different times. Further, any specific numbers noted herein are only examples, such that alternative implementations can employ differing values or ranges.

Details of the disclosed implementations can vary considerably in specific implementations while still being encompassed by the disclosed teachings. As noted above, particular terminology used when describing features or aspects should not be taken to imply that the terminology is being redefined herein to be restricted to any specific characteristics, features, or aspects with which that terminology is associated. In general, the terms used in the following claims should not be construed to limit the disclosure to the specific examples disclosed herein, unless the Detailed Description above explicitly defines such terms. Accordingly, the actual scope encompasses not only the disclosed examples but also all equivalent ways of practicing or implementing the present disclosure under the claims. Some alternative implementations can include additional elements to those implementations described above or include fewer elements.

Any patents, applications, and other references noted above, and any that may be listed in accompanying filing papers, are incorporated herein by reference in their entireties, except for any subject matter disclaimers or disavowals, and except to the extent that the incorporated material is inconsistent with the express disclosure herein, in which case the language in this disclosure controls. Aspects can be modified to employ the systems, functions, and concepts of the various references described above to provide yet further implementations.

To reduce the number of claims, certain implementations are presented below in certain claim forms, but the applicant contemplates various aspects in other forms. For example, aspects of a claim can be recited in a means-plus-function form or in other forms, such as being embodied in a computer-readable medium. A claim intended to be interpreted as a means-plus-function claim will use the words “means for.” However, the use of the term “for” in any other context is not intended to invoke a similar interpretation. The applicant reserves the right to pursue such additional claim forms either in this application or in a continuing application.

What is claimed is:

1. A method for batch processing of medical studies, comprising:
receiving, by a computing system, input describing a plurality of medical studies to be carried out, wherein the input includes a query template and a set of variable parameters with corresponding values;
determining, by the computing system, a plurality of parameters for a plurality of jobs based on the input;
generating, by the computing system, the plurality of jobs based on the query template and combinations of the variable parameters, wherein each job corresponds to a distinct combination of parameter values;
executing, by the computing system, a subset of the plurality of jobs to produce results for each executed job;
performing, by the computing system, statistical analysis on the results of the executed jobs to identify jobs that produced statistically significant results;
based on the statistical analysis, determining, by the computing system, parameters or parameter values for subsequent jobs in the plurality of jobs; and
modifying at least one of the subsequent jobs or generating additional jobs to further explore parameters associated with the statistically significant results.

2. The method of claim 1, wherein the input comprises a natural language description of the plurality of medical studies, and wherein the method further comprises processing the natural language description using a large language model to determine the query template and the set of variable parameters.

3. The method of claim 1, wherein the statistically significant results comprise results having a p value below a threshold value.

4. The method of claim 3, wherein the threshold value is one of 0.05, 0.01, or 0.001.

5. The method of claim 1, wherein modifying at least one of the subsequent jobs comprises:
increasing a number of parameter value bins for a parameter determined to be a significant driver of outcome; or
eliminating a parameter determined to have statistical significance below a threshold value.

6. The method of claim 1, further comprising adjusting a prioritization of remaining jobs in the plurality of jobs based on the statistical analysis, wherein jobs that are more similar to the jobs that produced statistically significant results are prioritized for execution before other jobs.

7. The method of claim 1, further comprising:
utilizing a machine learning model to cluster studies with similar results; and
based on the clustering, identifying variables that are most likely to produce statistically significant results when varied.

8. The method of claim 7, wherein the machine learning model comprises a clustering algorithm trained to identify relevant parameters or trends in results of the executed jobs.

9. The method of claim 1, further comprising:
receiving a cap on a number of studies to be run or a total budget; and
automatically tuning the parameters and a number of jobs to fit within the cap or the total budget while optimizing study parameters so that jobs that are run are more likely to produce statistically significant results.

10. The method of claim 1, further comprising:
generating an output indicating summary statistics of the executed jobs;
identifying which jobs were determined to produce statistically significant results; and
determining general characteristics of the jobs that produced statistically significant results.

11. A system for batch processing of medical studies, comprising:
one or more processors; and
a memory storing instructions that, when executed by the one or more processors, cause the system to:
receive input describing a plurality of medical studies to be carried out, wherein the input includes a query template specifying syntax for queries to be executed and a combinations specification defining variable parameters and corresponding values for the variable parameters;
generate a plurality of jobs based on the query template and the combinations specification, wherein each job corresponds to a distinct combination of values for the variable parameters;
execute a subset of the plurality of jobs against one or more data sources containing medical data to produce results for each executed job;
analyze the results of the executed jobs to determine which jobs produced statistically significant results based on a threshold p-value; and
adjust prioritization of remaining jobs in the plurality of jobs based on the analysis, wherein jobs that are more similar to jobs that produced statistically significant results are prioritized for execution before jobs that are less similar to jobs that produced statistically significant results.

12. The system of claim 11, wherein the instructions further cause the system to modify at least one of the remaining jobs based on the analysis by increasing a number of parameter value bins for a parameter determined to be a significant driver of outcome.

13. The system of claim 11, wherein the instructions further cause the system to modify at least one of the remaining jobs based on the analysis by eliminating a parameter determined to have no statistical significance across the executed jobs.

14. The system of claim 11, wherein the instructions further cause the system to generate additional jobs to further explore parameters associated with the statistically significant results.

15. The system of claim 14, wherein generating additional jobs comprises creating jobs with finer-grained variations of parameter values for parameters identified as significant drivers of outcome.

16. The system of claim 11, wherein the instructions further cause the system to:
receive a cap on a number of jobs to be executed or a total budget; and
automatically tune the variable parameters and a number of jobs to fit within the cap or the total budget while optimizing study parameters so that jobs that are executed are more likely to produce statistically significant results compared to randomly selecting jobs.

17. The system of claim 11, wherein the instructions further cause the system to utilize a machine learning model to identify patterns across results of the executed jobs, wherein the machine learning model comprises a clustering algorithm configured to cluster studies with similar results.

18. The system of claim 17, wherein the instructions further cause the system to utilize the machine learning model to identify variables that are least likely to produce statistically significant results when varied and to determine that jobs involving such variables should not be executed.

19. The system of claim 11, wherein the one or more data sources containing medical data comprise at least one of electronic health records, claims data, pharmacy data, or public health tracking databases.

20. The system of claim 11, wherein the instructions further cause the system to generate an output indicating summary statistics of the executed jobs, identification of which jobs produced statistically significant results, and general characteristics of the jobs that produced statistically significant results.
